(19) United States
(12) Patent Application Publication
(10) Pub. No.: US 2019/0344017 A1
(43) Pub. Date: Nov. 14, 2019

(54) CLOSURE PIECE FOR A CONTAINER FOR PHARMACEUTICAL PREPARATIONS, AND CONTAINER PROVIDED WITH THIS CLOSURE PIECE

(71) Applicant: Schott Schweiz AG, St. Gallen (CH)

(72) Inventor: Mustafa Kücük, Staad (CH)

(73) Assignee: Schott Schweiz AG, St. Gallen (CH)

(21) Appl. No.: 16/411,833

(22) Filed: May 14, 2019

(30) Foreign Application Priority Data
May 14, 2018 (DE) 10 2018 111 449.4
May 14, 2018 (DE) 20 2018 102 680.1

(51) Int. Cl. A61M 5/31; B65B 7/28; B29C 45/00; A61J 1/14

(52) U.S. Cl. CPC A61M 5/31; B65B 7/2835; B29L 2031/7544; A61J 1/1418; B29C 2045/0027; B29C 45/0025

(57) ABSTRACT

A closure piece is provided for the releasable closure of an opening at the distal end of a cylindrical container for pharmaceutical preparations. The container may be a syringe with a Luer lock fastening. The closure piece includes a cylindrical closure cap, with a cylindrical cavity for receiving and retaining a sealing element, and a sealing element. The sealing element has a rearward offset in relation to an outer edge of the closure cap.

### BACKGROUND OF THE INVENTION

### 1. Field of the Invention

The present invention relates generally to a closure piece for a container for pharmaceutical preparations, and to a container for pharmaceutical preparations that is provided with this closure piece, in particular a syringe.

### 2. Description of the Related Art

Containers for pharmaceutical preparations, particularly syringes, play an important part in the administration of pharmaceutical preparations or medicaments. These containers, generally of cylindrical shape, comprise a chamber for receiving the preparation, the chamber being delimited at both sides by a proximal end and a distal end. A piston can be inserted into the proximal end and, in a sliding movement, can push the preparation in the distal direction through an opening at the distal end of the syringe or, in a reverse proximal movement, can draw a preparation into the syringe through this opening. The opening at the distal end is often formed by a conical tip.

Closure pieces or closure systems for closing the distal opening of the container have long been known. U.S. Pat. Nos. 5,624,402 and 6,196,998, for example, describe such closure pieces for closing a distal opening of a syringe. Further closure systems are described, for example in European Patent EP 1 600 190 B 1.

The known closure pieces or closure systems have several disadvantages.

Known closure pieces are often of a multi-part design and have an inner element made of a flexible material for sealing, and an outer element which creates the connection to the container. A screw connection is often provided for this purpose. The screwing-on of these multi-part closure pieces proves difficult if, as is known from the prior art, it is the inner element that first comes into contact with the container during the joining process. This can very easily lead to tilting or wedging at the start of the joining process, which stands in the way of the short processing times required for assembly of the closure piece.

Moreover, the inner element is often produced from an elastomeric material which, for production reasons, may be contaminated, for example with silicone oil. The closure pieces are generally stored and transported as bulk material. If this partially contaminated elastomeric material of the inner element now comes into contact with an outer thread of another closure piece or of a container, contamination of this component may occur, which may be critical as regards the assembly of the closure piece and for its subsequent opening, since in some cases predefined forces and moments can no longer be safely maintained, such that the reliability of the connection and therefore of the entire product can no longer be ensured.

Accordingly, it would be desirable to have a closure piece, and a method for assembling this closure piece on the container, permitting contamination-free and safe assembly with short processing times.

Further disadvantages lie in the precision and characteristics of the tightening torque or opening torque when assembling the closure piece or when releasing the closure piece from the container. Particularly during the release, i.e. when opening the container, known broad thread turns of the closure piece can lead to a constantly high opening torque, which the user has to overcome.

It would therefore be desirable to make available a closure piece with an opening torque which makes it possible to precisely maintain a predefined threshold value for opening the closure piece but at the same time simplifies the subsequent release.

Further problems lie in the high degree of precision of the required distances, in the assembled position, between the components that are required for sealing. The axial distance by which the element provided for sealing is separated from the opening of the container that is to be closed is of great importance here. The given shape and position tolerances can have the effect that a required minimum pressing force for the sealing cannot be maintained, as a result of which the reliability of the product may be called into question.

It would therefore be desirable to make available a closure piece with which it is possible to safely ensure the required minimum pressing force for sealing the opening of the container.

Further disadvantages lie in the color stability of the material of the closure piece, which often comprises plastic. This color stability is important in the sense that the closure piece and/or the container provided with a closure piece may be subjected to sterilization treatment, which can lead to color changes or other changes of the properties of the plastics involved.

It would therefore be desirable to make available a closure piece which allows the customary sterilization methods to be used without causing a visible change of color or any other change of the closure piece.

### SUMMARY OF THE INVENTION

Exemplary embodiments disclosed herein provide a closure piece for a cylindrical container for pharmaceutical preparations, in particular a syringe, and also a container for pharmaceutical preparations that is provided with this closure piece, in particular a syringe.

The closure piece provided according to the invention avoids the abovementioned disadvantages and therefore affords several design improvements in relation to known closure pieces or closure systems.

Cylindrical containers for pharmaceutical preparations are generally elongate and have a central chamber with a proximal end which adjoins the chamber on one side and into which a piston for example can be inserted, and an opposite distal end which can comprise a conically tapering tip. The central axis of the container constitutes the longitudinal axis of the arrangements described further herein.

The distal end is generally of an elongate shape tapering conically towards the distal end and has an opening in the form of a narrow passage through which, for example, liquid pharmaceutical preparations can be driven out of the chamber if a suitable piston movement takes place. Conversely, liquid preparations can also be drawn in, if the piston is moved in the opposite direction, i.e. in the direction of the proximal end. In the pharmaceutical field, these cylindrical containers are known as syringes or cannulas and can be made of glass or plastic. The closure piece provided according to the invention is in principle suitable both for containers made of glass and containers made of plastic. The pharmaceutical preparations can comprise liquids, gases, solids or mixtures of these.

In order to secure a needle cannula, for example, at the distal end of the container, a device referred to as a Luer lock has become established in the medical field. In the case of containers made of plastic, this Luer lock fastening can be formed in one piece with the container, for example as a sleeve or collar, and can at least partially enclose the tip of the container. This Luer lock fastening can comprise a sleeve with an inner thread at the distal end of the container.

To seal the opening at the distal end of the container or to close the passage of the tip, a closure piece is needed, for example, in order to avoid contamination of filled preparations or in order to counteract accidental loss of the preparation. The term “sealing” or “closing” is understood further herein as a pressure-tight and/or fluid-tight closure of the opening of the distal end of the container.

Exemplary embodiments disclosed herein accordingly provide a closure piece for the releasable closure of an opening at the distal end of a cylindrical container for pharmaceutical preparations, in particular a syringe, such as by a Luer lock fastening.

The closure piece provided according to the invention may be used for closing the opening of a distal end of the container that is configured as a tip. The container may be made of plastic and comprise a sleeve with a Luer lock fastening with an inner thread.

In some exemplary embodiments, the provided closure piece comprises: a cylindrical closure cap, with a cylindrical cavity for receiving and retaining a sealing element, the closure cap comprising a mounting portion which, in the mounted position, faces the container, a sealing element with a sealing portion, which is arranged at least partially inside the mounting portion, the outer edge of the sealing element having a rearward offset in relation to the outer edge of the mounting portion.

Here, the outer edge of the sealing element means the outer edge of the sealing element facing towards the container in the assembled position; the outer edge of the mounting portion likewise means the outer edge of the closure piece facing towards the container in the assembled position.

The closure cap can have a one-part or multi-part design and, in some embodiments, has a mounting portion, which is designed for connection to the container and thus faces towards the container in the assembled position, and an actuating portion, which is arranged opposite and allows a user to grip the closure piece.

If the container has a Luer lock fastening with an inner thread, the mounting portion can comprise an outer thread which, in the assembled position, can engage in the Luer lock fastening of the container and in this way can create a releasable connection. In the assembled position, closure cap and container are arranged coaxially with respect to the longitudinal axis. The closure piece provided according to the invention, with the sealing element inserted in the closure cap, can be suitable for mounting on a container with a Luer lock fastening that comprises an inner thread for receiving an outer thread providing a mirror-inverted match.

The actuating portion can be equipped with outer ribs for better handling by the user. The cylindrical cavity of the closure cap can be of a continuous configuration for simple placement and retention of a, for example, one-part sealing element, which can likewise be cylindrical and can have an outer geometry providing a mirror-inverted match to the inner geometry of the cylindrical cavity of the closure cap. The sealing element may be produced from an elastomeric material.

The rearward offset according to the invention makes it possible, in a surprisingly simple way, to considerably improve the assembly process and to increase the process safety, since tilting and wedging during placement of the closure piece onto the container can be effectively avoided. This is due to the fact that, during the joining process, the closure piece is first of all centered via the mounting portion, before the sealing element comes into contact with the container, in particular with the tip of the container. Specifically in fully automated assembly processes, and with the high piece numbers required, an improvement in the safety of assembly is of great importance for the efficiency of production.

The rearward offset of the sealing element in relation to the outer edge of the mounting portion of the closure cap, in other words a sealing element recessed in relation to the outer edge on the assembly side of the closure piece, thus increases the process safety, since centering of the closure piece takes place in the screwing-in procedure even before the sealing element comes into contact with the container. By contrast, closure pieces available on the market have a sealing element which protrudes from the closure cap in the direction of assembly. The initial contact is therefore between sealing element and container during assembly, which makes it difficult or even impossible to achieve the desired centering.

In the present case, the first contact, when joining the sealing element to the tip of the container, takes place only after closure piece and container have been centered, such that a purely axial pushing-on of the sealing element is possible, which is less susceptible to error.

At the same time, the rearward offset of the sealing element also leads to a reduction of the contamination of other closure pieces or also of the container, for example by manufacturing residues that can accumulate on the surface of the sealing element. For example, these can involve liquids used in the manufacture of the sealing elements, for example silicone oil, which can accumulate on the outside of the sealing element. This is often the case specifically in sealing elements that are produced from an elastomeric material.

The closure pieces with the sealing element are often stored, transported and delivered as bulk material, with several thousand of them in one package, for example a bag. If the sealing element protrudes from the closure piece, it may, if contaminated, also contaminate other closure pieces in the package if it touches them. For example, silicone-treated sealing elements, i.e. sealing elements contaminated with silicone oil, arranged in the package may touch other closure pieces that are not treated with silicone. Through this contact, a closure piece not treated with silicone may be contaminated with silicone oil.

If, for example, an outer thread of another closure piece is contaminated with silicone oil, this contamination is disadvantageous during transport or also in subsequent processes such as singulation, gripping, placing the closure piece onto the container at the appropriate machine. By virtue of the rearward offset of the sealing element, this undesired contamination can be counteracted.

As regards the product too, contamination of the outer thread of the closure cap brings disadvantages, for example in terms of the tightening torque needed for assembly and in terms of the releasing or opening torque that is later needed for release. If, for example, a contaminated sealing element comes into contact with an outer thread, the tightening torque may be substantially reduced or it may be subject to strong variations.

As regards the container, reducing the danger of contamination can also increase the reliability of the container closed with the closure piece, provided that it is possible to safely rule out the possibility of manufacturing residues of the sealing element settling on a thread during the course of the production process and/or assembly process.

In tests, for example, it was possible to demonstrate that the tightening torque and the opening torque begin to vary strongly as a result of contamination of the outer thread. This means that a predefined moment can no longer be maintained. The measured variance of the tightening torque in the case of contaminated closure pieces can be more than 0.5 N*cm, more than 1.0 N*cm or even more than 1.5 N*cm. The variance of the opening torques in the case of contaminated closure pieces can be between 2 and 16 N*cm, between 2 and 18 N*cm or even between 2 and 20 N*cm. Such strong variations are disadvantageous for a user.

It is therefore also advantageous, on the opposite side of the closure cap, to provide such a rearward offset of the sealing element relative to the outer edge of the actuating portion in order to reduce the danger of contamination.

Exemplary embodiments provided in accordance with the invention therefore not only make a contribution to increasing the process safety but also to increasing the product reliability of the containers for pharmaceutical preparations. A closure piece can be made available in which the tightening torque and/or the opening torque have a slight variance of +/−1.0 N*cm, such as of +/−0.5 N*cm, in relation to the respectively required or desired torque.

This is achieved by the fact that the length of the sealing element is such that the potentially contaminated sealing elements cannot touch other closure caps. The length of the sealing element along the longitudinal axis is therefore smaller than the length of the closure cap. In relation to the length of the closure cap, the length of the sealing element in some embodiments is at most 95%, such as at most 90% or at most 85%.

The assembly and disassembly behavior of the closure cap is to a large extent determined by the design of the outer thread of the closure cap. If the container has a Luer lock fastening, a special design of the outer thread of the closure cap can permit easier and more precise assembly, which can likewise increase the process safety and also the product reliability.

For this purpose, the outer thread of the closure cap can be configured according to the current standard ISO 594-2, although in some embodiments a special design of the thread, such as another shape of the thread flank, is provided which leads to another engagement of the outer thread in the inner thread of a Luer lock attachment. In some embodiments, provision is made for the thread flank to be narrower and/or have a steeper flank, which with the same pitch leads to a greater distance of the thread flanks from each other and therefore to a slight axial play of the closure cap only partially screwed into a Luer lock attachment.

The thread may be configured as a trapezoidal thread, with a thread crest width of approximately 0.6 mm and a maximum width of base of approximately 1.2 mm. This leads initially to easier screwing of the closure piece onto the container, since the acting frictional forces are lower. Other design variables of the thread may remain the same, for example the thread pitch.

An exemplary thread pitch within the meaning of the invention is between 3 and 5.5 mm, such as between 3.5 and 5.25 mm or between 4 and 5 mm.

This also makes it possible to provide an elevation at at least one defined position on the thread flank, such as a punctiform elevation, which for example protrudes by between 0.5 and 1.5 mm for the flank. In some embodiments, at least two elevations are provided which are arranged at opposite positions of the thread flank. Two elevations arranged opposite each other can thus form what is called an elevation pairing. Such an elevation pairing may be arranged in such a way that, in the assembled position, the elevation points then bear on the thread flank of the Luer lock fastening and are slightly compressed or deformed thereby. An additional force component is thus introduced into the connection between closure piece and container. Compared to a single elevation, an elevation pairing affords the advantage that this additional force component does not act just on one side.

This force component equally acts also in the opening of the closure piece and thus makes it possible to precisely provide a predefined threshold value for opening the closure piece. In this way, a better characteristic, easier for the user, can be made available for the opening torque for releasing the closure piece from the container.

For a uniform introduction of force, more than one elevation pairing may be provided, such as two or four elevation pairings. In this way, a contribution can be made towards a uniform and firm fit of the closure piece on the container. This also promotes a precise adjustment of the torque required for the assembly and/or release, and it also ensures a predetermined force profile during the rotation movement.

Generally, when mounting a closure piece onto containers that are used for pharmaceutical preparations, it is essential to comply precisely with prescribed forces, since exceeding or falling below these values can quickly have an unfavorable effect on the product safety.

By the outer thread of the closure cap configured according to the invention, it is possible to maintain a very tight assembly torque, for example 18.0 N*cm at a tolerance of +/−1.0 N*cm, such as of +/−0.5 N*cm. This is important, since a tightening torque of more than approximately 20 N*cm in Luer lock fastenings, particularly ones made of plastic, can lead to the formation of microfissures. This can lead to failure of the container and/or of the closure piece, as a result of which the product reliability is no longer ensured. If the tightening torque is less than 16 N*cm, the leaktightness in the case of tips with a low Luer lock resistance cannot really be guaranteed.

The special design of the thread has the further effect that, when opening the closure piece, a higher force component initially has to be applied for release, after which this force component constantly reduces. This makes it easier for the user to open the container and release the closure piece. The torque to be applied by the user accordingly depends on the opening angle, i.e. the orientation of closure piece to container. After an initial resistance at the start of the release procedure has been overcome, which resistance is provided by the additional force component on account of the compression of the elevation points, the acting force component decreases on account of the narrower thread turns which, after the release, afford more space to the elevation points, such that the elevations are less compressed or are no longer compressed. In this way, the torque that has to be applied for the further release drops back. It is thus possible to set a precise torque for the opening procedure, for example 16 N*cm with a precision of +/−0.5 N*cm.

Thus, the release behavior of the closure piece according to the invention also differs from known closure pieces in which the force components to be applied at the start of the release procedure barely fall during the release of the closure piece. This is due to the fact that known closure pieces often have a bulging of the thread turn for an additional force component, but the latter then acts throughout the release procedure because of the broader thread turn. For the user, this characteristic of the torque that has to be applied for release is somewhat unfavorable, since a higher moment has to be applied for almost the entire opening procedure.

According to the invention, the mounting of the closure piece is further simplified by the fact that the thread turn, as has been stated above, is made narrower, which increases and thus facilitates the possible angle orientation, i.e. the correct-angle alignment, of closure piece and container relative to each other at the start of assembly.

In order to further extend this tolerance range of the required angle orientation, in some embodiments the portion of the thread turn adjacent to the thread entry is provided with a bulge in order to increase the free space from the outer edge as far as the first thread turn, as a result of which the tolerance range of the angle orientation can be further increased. This bulge thus constitutes a slight axial offset of the thread turn in said portion. On account of the narrower thread turn, this bulge can therefore also be configured in such a way that no additional force component acts on the connection.

A narrow tolerance range for placement and screwing-in of the closure piece at the correct angle to the container is therefore not necessary. Instead, a greater angle range is provided within which the assembly can take place. Whereas in known closure pieces this tolerance range is given with respect to the correct-angle assembly, i.e. a deviation from the ideal orientation of less than +/−3°, the configuration of the thread entry according to the invention allows this angle tolerance to be extended to a range of +/−10° or even more. Depending on the specific configuration, this angle tolerance according to the invention can lie in a range of between +/−5° and +/−15°, such as between +/−8° and +/−12°. This leads to a much more favorable assembly behavior, since the effort for exact orientation of the closure piece with respect to the container can be reduced.

In some embodiments, the thread entry can additionally comprise a bevel or the thread entry can be configured as a bevel. Whereas known closure pieces have an outer thread with a blunt thread entry, which can very quickly lead to wedging during the start of the assembly procedure, this insertion bevel at the thread entry greatly facilitates assembly.

The reliability of the pharmaceutical preparations container provided with the closure piece provided according to the invention is further influenced by the precision fit of those components which effect the leaktightness of the opening of the container. Of crucial importance here is the axial spacing between a radially outwardly protruding stop of the closure cap, which stop bears on the container in the assembled position, in particular on the collar of the Luer lock fastening, and the inner edge of a sealing portion of the sealing element, which seals off the opening of the tip. In the assembled position, the sealing portion of the sealing element bears tightly on the opening, in particular on the tip, and/or encloses the opening, wherein a minimum pressing force is required, which can be set via the shape tolerance and position tolerance of the components involved.

For the axial positioning of the sealing element in the opening of the closure cap, the latter may be configured with a radially inwardly protruding peripheral shoulder. On its circumferential surface, the sealing element may have a likewise peripheral recess which can be positioned in such a way that, in the assembled position, the shoulder of the closure cap can engage in the recess of the sealing element and provide locking by this axial stop. In this way, it is possible to achieve a precise positioning of the sealing element, and therefore of the inner edge of the sealing element that seals off the opening of the container, in relation to the container in the assembled position.

For customary containers, in particular for customary syringes, an assembly dimension of 2.85 mm with a very small tolerance is required for the axial spacing between the outwardly protruding stop of the closure cap, also designated as outer stop, and the stop defined by the shoulder inside the opening of the closure cap, also designated as inner stop. The inner stop acts in an opposite direction with respect to the minimum pressing force of the sealing element that has to be applied for sealing and thus defines the sealing force acting on the opening of the tip.

Accordingly, for the configuration of the closure piece, it is thus of great importance that a very narrow tolerance for this inner stop and outer stop is maintained. According to the invention, this tolerance is maintained by various design measures.

On the one hand, in some embodiments, the closure cap is configured in such a way that a thin and above all uniform material thickness of the side wall is provided over the length of the closure cap. The wall thickness of the closure cap at the thickest point may be at most 2.5 mm, such as at most 2.0 mm. Viewed along the entire length of the closure cap, the thickness of the wall varies by not more than 0.5 mm, such as by not more than 0.3 mm.

Maintaining this dimension is not a trivial matter. The closure caps are generally made of thermoplastics by injection molding. This leads to higher temperatures in the shaping process, which can lead to dimensional discrepancies of the closure cap after cooling. A constant wall thickness leads to homogeneous cooling and thus reduces possible warpage.

Moreover, according to the invention, the injection molding method is optimized in the sense that an injection point is chosen that lies in the region of the actuating portion. The injection point may lie externally on the circumferential surface of the actuating portion at an axial distance from an outer rib. A possible risk of injury to a user can moreover be counteracted in this way, since sharp protruding edges may arise at the gate. By contrast, an injection point on one of the outer ribs is not preferred, since this may lead to warping of the thin-walled closure cap.

The method provided according to the invention allows the closure cap to be produced by injection molding in very high piece numbers and with a very narrow dimensional tolerance, which is partly due to the thin and uniform wall thickness and to the optimized position of the injection point.

To be able to further maintain the abovementioned narrow tolerance, it is moreover beneficial that the sealing element is retained in a precise manner in the closure cap. In particular, unintended settling movements of the sealing element in the axial direction relative to the closure cap are to be ruled out as far as possible, which movements can occur as a result of the placement and/or assembly.

The rotationally secure retention of the sealing element in the continuous opening of the closure cap can be obtained via at least two elongate, radially inwardly protruding inner ribs arranged axially in their main orientation on the inner wall of the opening, such as in the region of the actuating portion. Approximately 6 to 10 such inner ribs are typically provided, often also 8 regularly arranged inner ribs. To provide uniform pressing, these inner ribs can expediently be distributed at uniform intervals about the inner circumference. These inner ribs thus serve to counteract a rotation of the sealing element in the closure cap, for example during assembly or opening, and thus constitute an anti-rotation element.

Given that the closure cap is produced by injection molding, these inner ribs can have a demolding bevel in the longitudinal direction, i.e. in the axial direction. However, this inclination of the inner ribs gives rise to a force component which acts axially on the sealing element and which can result in an axial movement of the sealing element out of the intended position. Despite the inner stop, this movement can lead to an unwanted axial offset of the sealing element, by 0.3 mm or even more, in relation to the predetermined settling height.

This offset thus influences the above-described axial spacing between the stop of the closure cap and the inner edge of the sealing portion of the sealing element, such that the predetermined dimension cannot always be safely maintained. This can also have the effect that the minimum pressing force between the sealing element and the tip of the container is not achieved, such that the required leaktightness in some cases cannot be maintained and, consequently, the necessary reliability of the container cannot be ensured. When screw connections are used for pharmaceutical packages, a minimum requirement, for example, is that the connection has to be able to withstand at least a pressure of 3 bar for a duration of 30 seconds.

On the other hand, in some cases the required opening torque, for example of at least 2 N*cm for screw closures in the pharmaceutical field, cannot be maintained if the required spacing and the minimum pressing force are not maintained.

Whereas known closure pieces often have broad and rather flat inner ribs, the present disclosure proposes another, narrower configuration of the inner ribs which leads to a much smaller displacement volume at the sealing element, in particular with the same number of inner ribs. This can greatly reduce the tendency of a sealing element mounted in the closure cap to form an axial offset, and therefore the required spacing after settling of the sealing element can be precisely maintained. By contrast, a large displacement volume leads to strong compression of the sealing element and thus promotes the undesired axial offset.

The axial offset of the sealing element in the opening of the closure cap can be limited by the inner rib, configured according to the invention, to less than 0.3 mm, such as to less than 0.2 mm or to less than 0.15 mm.

For this purpose, the inner rib has a width of less than 1 mm, such as of less than 0.9 mm or of less than 0.8 mm. Moreover, the width or the cross-sectional geometry of the inner rib may be the same over a large part of the length of the inner rib, in particular over a length of at least 50% of the length of the inner rib, such as over a length of at least 60% or of at least 65%. This also greatly reduces the tendency of the sealing element towards an axial offset in the closure cap.

In this way, it is possible to maintain the necessary minimum pressing force for closing the opening. Thus, a closure cap can be made available in which the axial distance between the outer stop and the inner stop of the closure cap measures 2.85 mm with a tolerance of +/−0.2 mm, such as of +/−0.1 mm or of +/−0.05 mm. In this way, the required minimum pressing force for sealing the opening can be safely maintained.

Moreover, the actuating portion can comprise at least two elongate outer ribs which are arranged axially in their main extent and protrude radially outwards from the circumferential surface, said outer ribs offering a user a better grip and thus making screwing and unscrewing easier. It is advantageous here to provide narrow outer ribs which extend along almost the entire length of the actuating portion. In some embodiments, these outer ribs have a length, i.e. an extent in the axial direction, of at least 4.5 mm, such as of at least 5.0 mm. The height of these outer ribs, i.e. the radial protrusion from the circumferential surface, may be at least 0.3 mm, such that the user is provided with a stable grip surface and can thus open the closure piece more efficiently and more easily.

The reliability and the possible applications of the pharmaceutical preparations container provided with the closure piece provided according to the invention are further increased by the color stability of the container and in particular of the container provided with the closure piece provided according to the invention. Since closure pieces are typically produced in large piece numbers by injection molding, materials that can be used for this purpose are mainly suitable thermoplastics.

The color stability is of great importance in the sense that the closure piece and/or the container provided with the closure piece may be subjected to sterilization treatment, which can lead to color changes or other changes of the properties of the plastics involved. The sterilization serves to free pharmaceutical packages, for example containers with pharmaceutical preparations, of as far as possible all of the microorganisms contained in or adhering to them.

Various treatment methods for sterilization are available, for example sterilization with hot air. This is a physical sterilization method in which thermostable medical products are exposed to dry heat. To free the products of microbes such as bacteria, fungi, viruses or spores, temperatures of at least 180° C. are needed for a period of at least 30 minutes.

Moreover, autoclaving is known as the most commonly performed sterilization method. Here, steam at temperatures of 120°, for example, is used at 0.5 bar. Sensitive products may therefore remain longer in the autoclave than under hot air sterilization.

In addition, the steam is better distributed over the product surface, which allows the required sterilization temperature to be better obtained across the whole product.

Another known method is that of irradiation with electromagnetic radiation, for example gamma rays or X-rays. In what is called gamma sterilization, the pharmaceutical preparations are irradiated with gamma rays at a defined energy dose. When gamma rays or X-rays are used, the irradiation is generally performed with an energy dose in the range of 25-40 kGy.

Most of the known sterilization methods limit the choice of materials, for example the material of the closure piece, in the sense that defined effects, for example on the color or the consistency of the material, are minimized. In other words, irradiation of the closure piece can for example modify the polymer structure of the used plastic, which can subsequently lead to color changes. Moreover, certain sensitive thermoplastics may suffer under the high temperatures that are sometimes needed and, for example, can become brittle or likewise change color.

While a change of color caused by sterilization may be entirely desirable in some areas of use, it is assumed in the context of this invention that a change of color of the closure piece is undesirable. Regardless of the nature of the sterilization, i.e. when using at least one of the abovementioned sterilization methods, there should as far as possible be no color shift, i.e. no change of color of the closure piece. Within the meaning of the present invention, a change of color is then considered desirable if the color locus of the material after the sterilization deviates from the color locus before sterilization by a value ΔE, defined as color locus change in the CIE 1931 system, by more than ΔE=0.05. The color locus change ΔE is given by the following formula:

ΔE=\sqrt{(x²+y²)}

Therefore, in some embodiments provided according to the invention, the material chosen for the closure piece is one that barely changes as a result of the sterilization or does not change color.

This is achieved highly surprisingly through the choice of certain color pigments and antioxidants for the material for producing the closure piece. It has been found that a very specific material composition, comprising certain selected pigments which provide a green grey coloring of the closure piece, does not tend or at least barely tends to change color when one of the abovementioned sterilization methods is used. Instead, the original color is at least almost completely retained and there is no color shift. This color corresponds to the color RAL 7009 according to the RAL color system.

This surprising color-stable effect is achieved by the fact that a composition of coloring pigments was able to be found which gives plastic a polymer structure that is stable at elevated temperatures and also when exposed to electromagnetic radiation as indicated above. A material for producing the closure piece comprises the following pigments as elements for providing color:
titanium dioxide Kronos® 2233
carbon black PRINTEX® F 85
6975 titanium orange
VYNAMON® Green 600734.

A closure piece made of material comprising these coloring pigments is therefore also suitable for sterilization methods performed with gamma rays or X-rays. It has been found that such closure pieces, when exposed to radiation at an energy dose in a range of 25-40 kGy, do not lead to any color changes, or any color changes readily perceptible to the human eye, and can therefore be regarded as being color stable.

A material composition of this kind is also suitable for other sterilization methods, for example steam sterilization at up to 134° C. for a period of 10 minutes, and does not lead to any color changes, or any color changes readily perceptible to the human eye.

This is favorable since the choice of the sterilization method to be used is therefore largely independent of the material of the closure piece. Moreover, a combination of the abovementioned sterilization methods does not lead to a loss of leaktightness. In particular, after the sterilization, the closure piece has a leaktightness of up to 4 bar for 30 seconds and has an opening torque of less than 15 N*cm.

Finally, in a further aspect, the subject matter of the invention concerns a method for contamination-free, releasable closure of a distal end of a cylindrical container for pharmaceutical preparations, such as a syringe, in particular with a closure piece as described above.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 illustrates a longitudinal section through a closure piece according to the prior art;

FIG. 2 illustrates a longitudinal section through an exemplary embodiment of a closure piece provided according to the invention;

FIG. 3 illustrates a longitudinal section through a container for pharmaceutical preparations, which container has a Luer lock fastening, with a partially mounted closure piece;

FIG. 4 illustrates a profile of the torque when opening a closure piece provided according to the invention, in order to release it from the container;

FIG. 5 illustrates a profile of the torque when opening a closure piece known from the prior art, in order to release it from the container;

FIG. 6 illustrates a plan view of a detail of an exemplary embodiment of a closure piece provided according to the invention with a bulge;

FIG. 7 illustrates a longitudinal section through an exemplary embodiment of a closure piece provided according to the invention with a sealing element;

FIG. 8 illustrates a longitudinal section through an exemplary embodiment of a closure piece provided according to the invention without a sealing element; and

FIG. 9 illustrates an oblique view of an exemplary embodiment of a closure piece provided according to the invention.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

In the detailed description that follows, identical reference numbers have been used, for the sake of clarity, to denote essentially identical parts in or on the embodiments. For better illustration of the invention, however, the exemplary embodiments shown in the figures are not always drawn to scale.

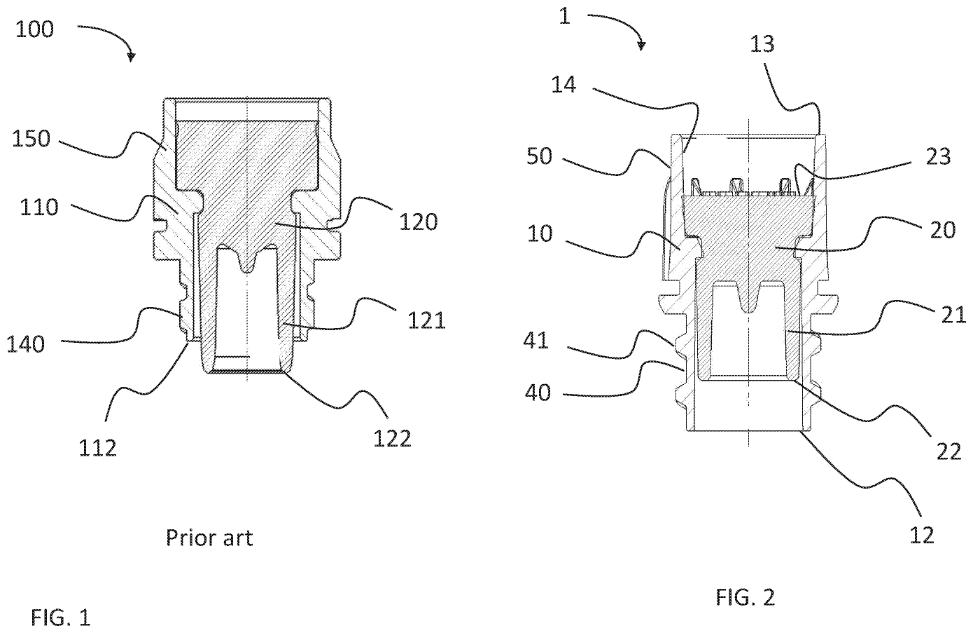

FIG. 1 shows a longitudinal section through a closure piece 100 according to the prior art. This closure piece 100 comprises a cylindrical closure cap 110 with a cylindrical cavity for receiving a sealing element 120, wherein the closure cap 110 comprises a mounting portion 140, which in the mounted position faces the container, and an actuating portion 150.

The sealing element 120, which has a sealing portion 121, is inserted into the cylindrical cavity of the closure cap 110. It will clearly be seen that the outer edge 122 of the sealing element protrudes past the outer edge 112 of the closure cap 110. This is unfavorable for automated assembly, since it can cause tilting and wedging at the start of the assembly procedure, when the sealing element first comes into contact with the container, in particular with the tip of the container. Thus, centering of the closure piece is also barely possible during the screwing-in procedure, since the sealing element 120 is produced from a resilient elastomeric material. Specifically in fully automated assembly processes and at the high piece numbers required, valuable time may be lost here if the assembly has to be carried out particularly slowly against this background or, for example after wedging, has to be repeated.

FIG. 2 shows a longitudinal section through a closure piece 1 provided according to the invention for the releasable closure of an opening at the distal end of a cylindrical container for pharmaceutical preparations (not shown), in particular a syringe, such as by a Luer lock fastening. The closure piece 1 provided according to the invention comprises a cylindrical closure cap 10 with a continuous cylindrical cavity 14 for receiving a sealing element 20, the closure cap 10 comprises a mounting portion 40, which in the mounted position faces the container, and an actuating portion 50.

The mounting portion 40 comprises an outer thread 41 which, in the mounted position, engages in the outer thread of the Luer lock fastening of the container and creates a releasable connection to the latter. The actuating portion 50 is equipped with outer ribs for handling by a user. The cylindrical cavity 14 of the closure cap 10 is of a continuous configuration for receiving the sealing element 20, which is in one piece in this example.

The figures make clear the pronounced rearward offset of the outer edge 22 of the sealing element 20 in relation to the outer edge 12 of the mounting portion 40 of the closure cap 10.

The rearward offset according to the invention makes it possible, in a surprisingly simple way, to greatly improve the assembly process and to enhance the process safety, since tilting or wedging can effectively be prevented when fitting the closure piece 1 onto the container or at the start of the assembly process. This is due to the fact that, during assembly, centering of the closure piece 1 first of all takes place via the mounting portion 40, before the sealing element 20 comes into contact with the container, in particular with the tip of the container. Specifically in fully automated assembly processes and at the high piece numbers required, an improvement in assembly safety is of great importance for the efficiency of production and can help to save valuable assembly time.

The rearward offset of the sealing element 20 in relation to the outer edge 12 of the mounting portion 40 of the closure cap 10 thus enhances the process safety, since centering of the closure piece 1 takes place during the screwing-in procedure even before the sealing element 20 comes into contact with the container. In the present case, the first contact of the sealing element 20 with the tip of the container thus takes place only after centering of closure piece 10 and container, such that the closure piece 10 can be pushed on in a purely axial movement, which is less susceptible to error.

At the same time, the rearward offset also leads to reduced contamination of other closure pieces, or also of the container, by manufacturing residues that may be present on or at the sealing element 20. These may be, for example, liquids used in the production of the sealing elements, for instance silicone oil. This is in fact often the case with sealing elements which, as here, are produced from an elastomeric material.

The rearward offset measures approximately 2.5 mm in the example. The danger of contamination can already be reduced with a rearward offset of at least 0.5 mm, such as of at least 1.0 mm or of at least 1.5 mm. In conjunction with the given tolerances, an exemplary rearward offset is moreover in a range of 1.5 to 2.0 mm, of 2.0 to 2.5 mm or of 2.5 to 3.0 mm.

It will also be seen from FIG. 2 that a rearward offset of the outer edge 23 of the sealing element 20 in relation to the outer edge 13 of the closure cap 10 is also provided. This rearward offset likewise may measure at least 0.5 mm, such as at least 1 mm or at least 1.5 mm. In the example, the rearward offset measures approximately 2.5 mm and is therefore approximately the same size as the rearward offset at the opposite side.

Accordingly, the rearward offset of the outer edges 22, 23 of the sealing element 20 in relation to the outer edges 12, 13 of the closure cap 10 reliably prevents mutual contamination of closure pieces lying next to one another, for example in transport packages. Structurally, this is achieved by a reduced length of the sealing element 20 relative to the length of the closure cap 10. In the example, the ratio of the length of the sealing element to the length of the closure cap is approximately 70%. A ratio of the length of the sealing element 20 to the length of the closure cap 10 of at most 95%, such as of at most 90% or of at most 85%.

The assembly and disassembly behavior of the closure piece 1 on the container is determined largely by the design of the outer thread of the closure cap 10.

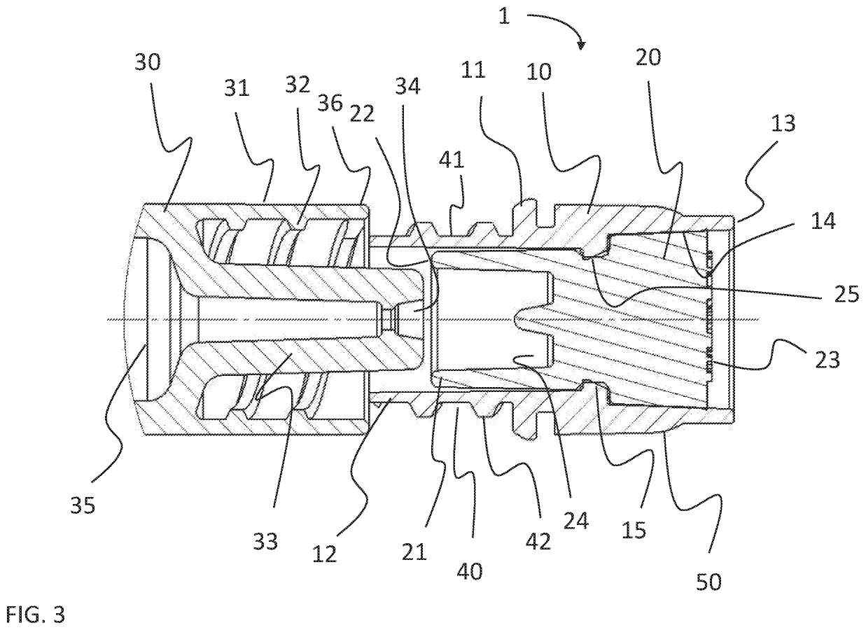

FIG. 3 shows a longitudinal section through a container 30 for pharmaceutical preparations that has a Luer lock fastening, with a partially mounted closure piece 1. At its distal end, the container 30 comprises a sleeve 31 which is configured with an inner thread 32 and constitutes the Luer lock fastening. The sleeve 31 encloses a conically tapering tip 33 at the distal end of the container, said tip having an opening 34, for example for discharging a preparation from the chamber 35.

The outer thread 41 of the mounting portion 40 is configured as an exact mirror-inverted match to the inner thread 32. The outer thread 41 is configured according to the current standard ISO 594-2, although another shape of the rib 42 is provided according to the invention. The rib 42 provided according to the invention is narrower and/or configured with a steeper flank, which leads to a narrower thread turn and therefore to a greater spacing of the adjacent thread flanks. This initially makes it easier to screw the closure piece 1 onto the container 30, since the acting frictional forces are lower.

This also makes it possible to provide elevations, such as punctiform elevations (not shown), at defined positions on the thread flank, which may be arranged in each case at two opposite positions of the thread flank. Two elevations arranged opposite each other can thus form an elevation pairing. Such an elevation pairing is configured in such a way that, in the assembled position, the elevation points than bear on the thread flank of the Luer lock fastening and are slightly compressed or deformed. This introduces an additional force component into the connection.

This force component also acts equally in the opening of the closure piece and thus makes it possible to precisely provide a predefined threshold value for opening the closure piece. In this way, a better characteristic, easier for the user, can be made available for the opening torque. In the example, the outer thread 41 comprises a total of four elevation pairings (not shown).

This also makes it easier to precisely set the torque needed for assembly and/or release and to ensure a predetermined force profile during the rotation movement.

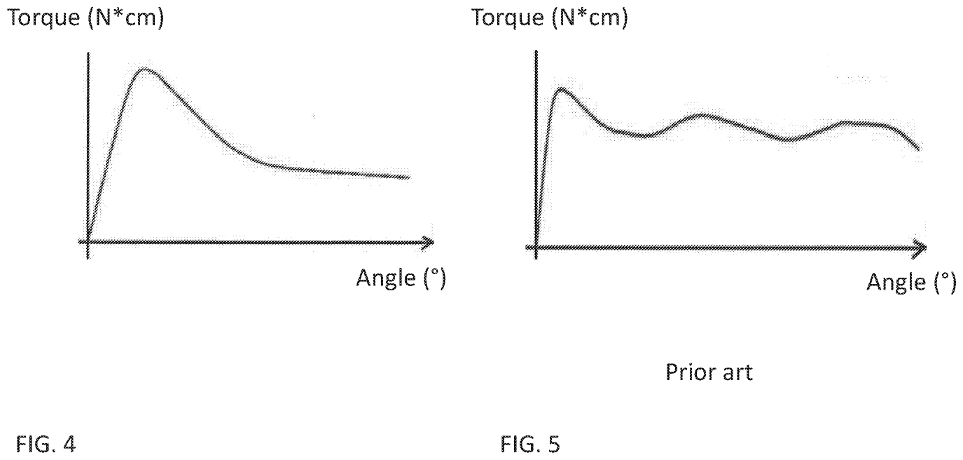

FIGS. 4 and 5 show, on the basis of illustrative embodiments, the profile of the torque when opening the closure piece 1 in order to release it from the container 30. FIG. 5 shows a characteristic curve for the opening torque of a closure piece 100 according to the prior art, while FIG. 4 shows the characteristic curve for the opening torque for a closure piece 1 provided according to the invention.

It will be seen that, when opening the closure piece 1 provided according to the invention, a higher force component has to be applied initially in order to release it. When a threshold value is exceeded, the force component to be introduced falls continuously. This makes it easier for the user to open the container 30 and release the closure piece 1. The torque to be applied is accordingly dependent on the opening angle. After overcoming an initial release resistance, which arises on account of the elevations, the acting force component decreases on account of the narrower thread turns which offer more free space, such that the elevations are less compressed. In this way, it is also possible to set a precise torque for opening, for example of 16 N*cm, with a precision of +/−0.5 N*cm.

The release behavior of the closure piece 1 provided according to the invention thus differs from closure pieces 100 known from the prior art in which the force component to be applied at the start of the release procedure hardly falls at all during the release of the closure piece and remains at a high level to the end. This can be seen clearly from the force profile in FIG. 5. This is due to the fact that the closure piece 100 provides a bulging of the thread turn for an additional force component, but the latter then acts throughout the release procedure. This characteristic curve of the torque that is to be applied for release is somewhat unfavorable for the user, since a higher moment has to be applied for almost the entire opening procedure.

According to the invention, the assembly of the closure piece is further simplified by the fact that the thread turn 41 is made narrower, which increases and thus facilitates the possible angle orientation of closure piece and container to each other at the start of the assembly. To further extend this tolerance range of the required angle orientation, in an exemplary embodiment the portion of the thread turn adjacent to the thread entry is provided with a bulge in order to increase the free space from the outer edge as far as the first thread turn, as a result of which the tolerance range of the angle orientation can be further increased.

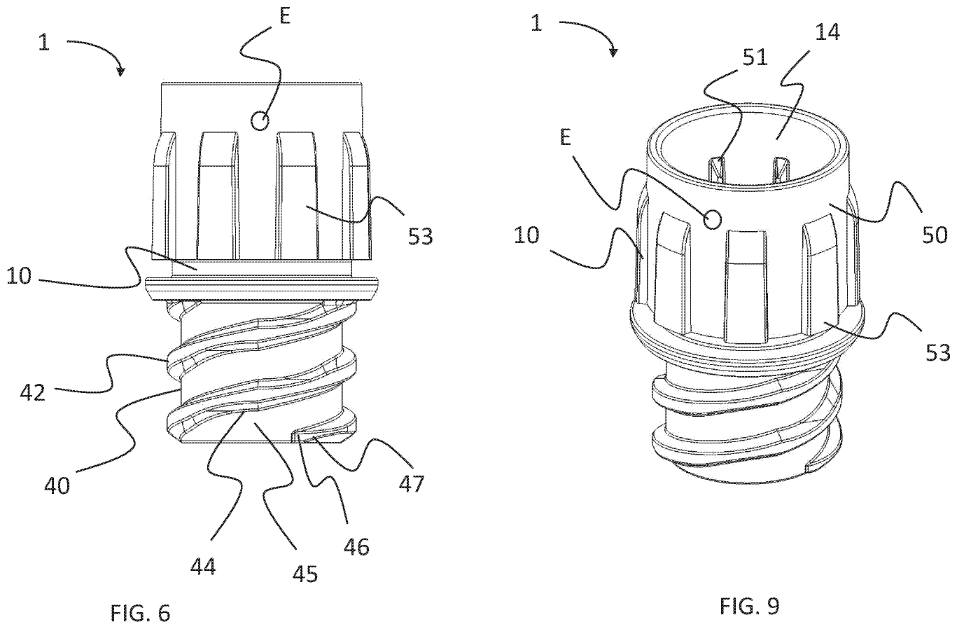

FIG. 6 shows a plan view of a detail of a closure piece 1 with a bulge 44. This slight axial offset of the thread turn, which constitutes the bulge 44, forms an additional free space 45 in a portion arranged adjacent to the thread entry. In this way, the tolerance range of the angle orientation is increased. In other words, the region which defines the required orientation of closure piece 1 and container 30 prior to joining is extended. The configuration, according to the invention, of the thread entry in the illustrative embodiment shown allows this angle tolerance to be extended to a range of +/−10°. According to the invention, it is possible to fix the angle tolerance in a range of between +/−5° and +/−15°, such as between +/−8° and +/−12°. This leads to a much more favorable assembly process.

In some embodiments, the thread entry 46 is additionally configured with a bevel 47 in order to further facilitate the joining together.

The reliability of the container 30 provided with the closure piece 1 provided according to the invention is further influenced by the precision fit of those components which effect the leaktightness of the opening 34 of the container 30. Of crucial importance here, as can be seen from FIG. 3, is the axial spacing between a radially outwardly protruding stop 11 of the closure cap 10, which stop 11 bears on the container 30 in the assembled position, in particular on the collar 36 of the Luer lock fastening 32, and the inner edge 24 of a sealing portion 21 of the sealing element 20, which effects the sealing of the opening 34 of the tip 33. In the assembled position, the sealing portion 21 bears tightly on the tip 33 and encloses the opening 34, wherein the inner edge 24 bears on the opening 34 with a predefined minimum pressing force.

For the axial positioning of the sealing element 20 in the opening 14 of the closure cap 10, the latter is configured with a radially inwardly protruding peripheral shoulder 15. On its circumferential surface, the sealing element 20 has a likewise peripheral recess 25 which is positioned in such a way that, in the assembled position, the shoulder 15 engages in the recess 25 and forms an axial stop. In this way, it is possible to achieve a precise positioning of the sealing element 20 and therefore of the inner edge 24 that seals the opening 34 of the container 30 (not shown).

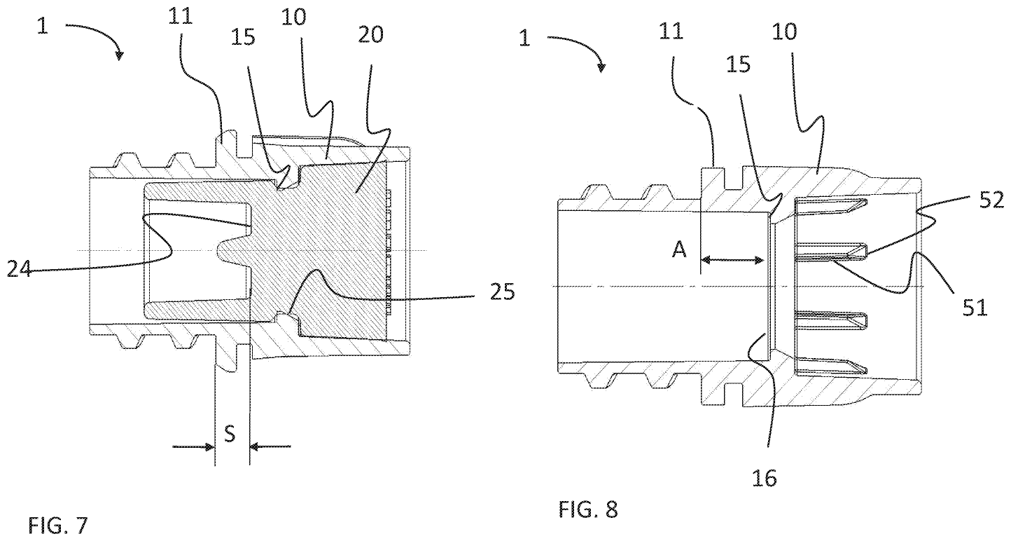

FIG. 7 shows these relationships on the basis of a longitudinal section through a closure piece 1 provided according to the invention with a sealing element 20. In this view, the letter S indicates the so-called settling, i.e. the distance which arises between the inner edge 24 of the sealing element 20 and the container 30 in the assembled position of the closure piece. This is decisive for obtaining the required minimum pressing force in order to seal the opening.

FIG. 8 illustrates these relationships once again on the basis of a longitudinal section through a closure piece 1 provided according to the invention, which in this view has no sealing element 20. To maintain this settling, a defined distance A is required between the outer edge of the outwardly protruding stop 11 of the closure cap 10, which therefore bears on the collar of the container in the assembled position, and a stop 16, defined by the shoulder 15, inside the opening 14 of the closure cap 10. In the example, an assembly distance of A=2.85 mm is required here with a very low tolerance. The inner stop 16 exerts on the opening the minimum pressing force to be applied by the sealing element 20 in order to permit sealing.

The closure cap 10 is configured in such a way that a thin and above all uniform material thickness of the side wall is provided over as far as possible the entire length of the closure cap, in order to achieve a high degree of dimensional accuracy. Therefore, the wall thickness of the closure cap 10 at the thickest point is at most 2.0 mm. Viewed along the entire length of the closure cap, the thickness of the wall varies by not more than 0.5 mm, such as by not more than 0.3 mm.

Moreover, according to the invention, the injection molding method is optimized in the sense that an injection point is chosen that lies in the region of the actuating portion 50. FIG. 9 shows an oblique view of a closure piece 1 provided according to the invention, wherein the injection point for the injection molding method is identified by the letter E in the example shown. The injection point thus lies externally on the circumferential surface of the actuating portion 50 at an axial distance from an outer rib 53. A possible risk of injury to a user can moreover be counteracted in this way, since sharp protruding edges may arise at the gate. In the present case, these potential gate edges lie outside the grip region.

The rotationally secure retention of the sealing element 20 in the continuous opening 14 of the closure cap is obtained via elongate, radially inwardly protruding inner ribs 51 arranged axially in their main orientation on the inner wall of the opening 14 of the actuating portion 50. To provide uniform pressing, these inner ribs 51 are distributed at uniform intervals about the inner circumference. In the example shown, a total of eight such inner ribs 51 are provided.

According to the invention, a narrow configuration of the inner ribs 51 is provided which leads to a much smaller displacement volume at an inserted sealing element 20. This greatly reduces the tendency of a sealing element 20 mounted in the closure cap 10 to form an axial offset, and therefore the required spacing after placement of the sealing element, i.e. the settling for maintaining the minimum pressing force, can be precisely maintained. The axial offset of the sealing element in the opening of the closure cap is limited by the inner rib, configured according to the invention, to less than 0.3 mm, such as to less than 0.2 mm or to less than 0.15 mm.

In the example shown, the inner rib 51 therefore has a width of approximately 0.9 mm. Without restriction to the illustrative embodiment, widths of the inner rib 51 of less than 1 mm, such as of less than 0.9 mm or of less than 0.8 mm, are expedient. Moreover, for a stable fit, the width and the cross-sectional shape of the inner rib 51 are constant over a large part of the length of the inner rib 51, as can be seen clearly from FIG. 8, for example. In other words, except for a small insertion bevel 52 for setting the sealing element, the cross section of the rib is constant along its length. It has proven expedient if the cross-sectional shape of the inner rib 51 is the same over a length of at least 50% of the total length of the inner rib 51, such as over a length of at least 60% or of at least 65%. This greatly reduces the tendency of the sealing element 20 towards a subsequent axial offset.

In this way, it is possible to maintain the necessary minimum pressing force for closing the opening 34. Thus, a closure cap 10 is made available in which the axial distance between the outer stop 11 and the inner stop of the closure cap measures 2.85 mm with a tolerance of +/−0.2 mm, such as of +/−0.1 mm or of +/−0.05 mm. In this way, the required minimum pressing force for sealing the opening 34 is safely maintained.

Moreover, it is particularly expedient if the actuating portion 50 comprises elongate outer ribs 53 which are arranged axially in their main extent and protrude radially outwards from the circumferential surface, said outer ribs 53 offering a user a better grip and thus making screwing and unscrewing easier. In the example, the actuating portion 50 comprises eight such outer ribs 53. These outer ribs 53 extend along almost the entire length of the actuating portion 50. In some embodiments, these outer ribs have a length, i.e. an extent in the axial direction, of at least 4.5 mm, such as of at least 5.0 mm. In the example, the length is approximately 5.3 mm, which already affords a sufficient grip surface.

The height of these outer ribs 53, i.e. the radial protrusion from the circumferential surface, is at least 0.3 mm, such that the user is provided with a stable grip surface and can thus open the closure piece more efficiently and more easily.

The closure piece 1 is made of a color-stable plastic. In order to color the plastic, the pigments listed below are used as elements for providing color:
titanium dioxide Kronos® 2233
carbon black PRINTEX® F 85
6975 titanium orange
VYNAMON® Green 600734.

A closure piece 1 made of such a material is therefore also suitable for sterilization methods performed with gamma rays or X-rays. It has been found that such closure pieces, when exposed to radiation at an energy dose in a range of 25-40 kGy, do not lead to any color changes, or any color changes readily perceptible to the human eye, and can therefore be regarded as being color stable.

A material composition of this kind is also suitable for other sterilization methods, for example steam sterilization at up to 134° C. for a period of 10 minutes, which does not lead to any color changes, or any color changes readily perceptible to the human eye.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### LIST OF REFERENCE SIGNS

1 closure piece
10 closure cap
11 stop
12 outer edge of the closure cap
13 outer edge of the closure cap
14 cavity
15 shoulder
16 stop
20 sealing element
21 sealing portion
22 outer edge of the sealing portion
23 outer edge of the sealing portion
24 inner edge
25 recess
30 container
31 sleeve
33 tip
34 opening
35 chamber
36 collar
40 mounting portion
41 outer thread
42 rib
44 bulge
45 free space
46 thread entry
47 bevel
50 actuating portion
51 inner rib
52 insertion bevel
53 outer rib
100 closure piece
110 closure cap
112 outer edge of the closure cap
120 sealing element
121 sealing portion
122 outer edge of the sealing portion
140 mounting portion
150 actuating portion
S settling
A distance
E injection point

What is claimed is:

1. A closure piece for the releasable closure of an opening at a distal end of a cylindrical container for pharmaceutical preparations, the closure piece comprising:
a cylindrical closure cap having a cylindrical cavity configured to receive and retain a sealing element, the closure cap comprising a mounting portion which, in a mounted position, faces the container; and
a sealing element with a sealing portion, which is arranged at least partially inside the mounting portion, an outer edge of the sealing element having a rearward offset in relation to an outer edge of the mounting portion.

2. The closure piece of claim 1, wherein the rearward offset of the outer edge of the sealing element in relation to the outer edge of the mounting portion measures at least 0.5 mm.

3. The closure piece of claim 1, wherein the closure cap further comprises an actuating portion arranged opposite the mounting portion, wherein the sealing element is arranged at least partially inside the actuating portion, and wherein the outer edge of the sealing element has a rearward offset in relation to an outer edge of the actuating portion.

4. The closure piece of claim 3, wherein an injection point is arranged at a position on a circumferential surface of the actuating portion of the closure cap at an axial distance from an outer rib.

5. The closure piece of claim 3, wherein the actuating portion comprises at least two elongate outer ribs arranged axially in their main orientation and protruding radially outwards from a circumferential surface of the actuating portion.

6. The closure piece of claim 1, wherein a ratio of a length of the sealing element to a length of the closure cap is at most 95%.

7. The closure piece of claim 1, wherein the sealing element comprises an elastomeric material.

8. The closure piece of claim 1, wherein the closure cap has an outer thread configured to connect to a Luer lock fastening of the container, wherein the outer thread is configured according to the standard ISO 594-2.

9. The closure piece of claim 8, wherein the outer thread is configured as a trapezoidal thread.

10. The closure piece of claim 9, wherein at least one punctiform elevation is provided on a thread flank of a thread turn of the outer thread.

11. The closure piece of claim 10, wherein an axial bulge is provided on a portion of the thread turn lying next to a thread entry.

12. The closure piece of claim 10, wherein the thread turn comprises a bevel.

13. The closure piece of claim 1, wherein the closure cap has a continuous opening, wherein a wall thickness of the closure cap at least one of is at most 1.5 mm at a thickest point or varies by not more than 0.5 mm as seen along a length of the closure cap.

14. The closure piece of claim 13, wherein the continuous opening of the closure cap has at least two elongate inner ribs arranged axially in their main orientation and protruding inwardly on an inner wall, wherein the inner ribs have a width of less than 1 mm.

15. The closure piece of claim 14, wherein the width or a cross section of the at least two inner ribs is constant over at least 50% of a length of the at least two inner ribs.

16. The closure piece of claim 1, wherein an axial distance between an outer stop and an inner stop of the closure cap measures 2.85 mm with a tolerance of +/−0.2 mm.

17. The closure piece of claim 1, wherein the closure piece comprises a color-stable plastic.

18. The closure piece of claim 17, wherein a color of the closure piece is green grey and corresponds to the tone RAL 7009 according to the RAL color system.

19. A method for contamination-free, releasable closure of a distal end of a cylindrical container for pharmaceutical preparations, the method comprising:
closing the distal end of the container with a closure piece, the closure piece comprising:
a cylindrical closure cap having a cylindrical cavity configured to receive and retain a sealing element, the closure cap comprising a mounting portion which, in a mounted position, faces the container; and
a sealing element with a sealing portion, which is arranged at least partially inside the mounting portion, an outer edge of the sealing element having a rearward offset in relation to an outer edge of the mounting portion.

20. The method of claim 19, wherein at least one of a tightening torque or an opening torque has a slight variance of +/−1.0 N*cm in relation to a respectively required torque.

21. A method for producing a closure piece, the closure piece comprising a cylindrical closure cap having a cylindrical cavity configured to receive and retain a sealing element, the closure cap comprising a mounting portion which, in a mounted position, faces the container, a sealing element with a sealing portion, which is arranged at least partially inside the mounting portion, an outer edge of the sealing element having a rearward offset in relation to an outer edge of the mounting portion, and an actuating element with at least two outer ribs, the method comprising:
injection molding the closure piece, wherein an injection point is chosen that lies in a region of an actuating portion at a position on an outer circumferential surface of the actuating portion at an axial distance from an outer rib.
